(19) United States
(12) Patent Application Publication
(10) Pub. No.: US 2023/0248442 A1
(43) Pub. Date: Aug. 10, 2023

(54) SYSTEM AND METHOD FOR CARDIAC STRUCTURE TRACKING

(71) Applicant: UNIVERSITY OF SYDNEY, SYDNEY, New South Wales (AU)

(72) Inventors: Nicholas HINDLEY, Sydney, New South Wales (AU); Paul KEALL, Sydney, New South Wales (AU); Chun-Chien SHIEH, Sydney, New South Wales (AU); Suzanne LYDIARD, Sydney, New South Wales (AU)

(21) Appl. No.: 18/014,691

(22) PCT Filed: Jul. 8, 2021
(86) PCT No.: PCT/AU2021/050729

§ 371 Date: Jan. 5, 2023

(30) Foreign Application Priority Data
Jul. 9, 2020 (AU) 2020902370

(51) Int. Cl. A61B 34/20; A61N 5/10; G06T 7/11

(52) U.S. Cl. CPC A61B 34/20; A61N 5/1037; G06T 7/11; A61B 2034/2046; G06T 2207/10076

(57) ABSTRACT

Systems, methods, and apparatus are disclosed for cardiac structure tracking. An example method includes segmenting a diaphragm or respiratory surrogate, heart, and target. The method also includes performing a peak-exhale to peak-inhale registration and generating a respiratory motion model. The method further includes tracking the diaphragm using X-ray imaging and estimating a target position for an x-ray guided cardiac radioablation treatment. The example method provides directly, precisely controlled x-ray guided cardiac radioablation that accurately targets the substrates of cardiac ablation while minimizing doses to healthy tissue.

### TECHNICAL FIELD

The present disclosure generally relates to systems and tracking methods for cardiac structure, and in particular, to systems and methods for cardiac substructure tracking during ablative radiotherapy.

### BACKGROUND

Cardiac arrhythmias represent a significant and growing health burden worldwide. Between 1990 and 2013, the number of deaths due to atrial fibrillation (AF) and atrial flutter rose from 29,000 to 112,000. Furthermore, atrial fibrillation was estimated to affect 2-3% of the world’s population with 1 in 4 people developing the disease over their lifetime. Similarly, in 2008, sudden cardiac death accounted for 15% of all deaths globally, for which 80% occurred due to ventricular tachycardia (VT). Regardless of whether aberrant electrical conductivity occurs in the atria or ventricles, the effects of cardiac dysregulation can be life-threatening.

The standard treatment for AF is catheter ablation, which typically involves guiding thin, flexible tubing to a patient’s heart where an arrhythmia is induced and abnormal tissue is ablated using local heating or freezing. This is a long and complex procedure, for which the effectiveness is largely dependent on the severity of disease as well as the experience of the operator. For patients with VT, catheter ablation has been shown to yield better outcomes than escalation of antiarrhythmic drugs and has been used to prevent the need for defibrillator therapy. However, substrates for VT ablation for are often deeper than those for AF, and thus present even greater challenges for catheter treatment. Furthermore, arrhythmias more commonly occur in the elderly, for whom there are often comorbidities that would exclude the use of such invasive procedures.

Radiotherapy has recently emerged as a non-invasive alternative to catheter ablation. In 2017, Cuculich et al. demonstrated the use stereotactic body radiation therapy (SBRT) to treat five patients with high-risk, refractory VT. This technique was shown to reduce the number of VT episodes from 6577, during the 15 patient-months prior to treatment, to 4 over the 46 patient-months after a 6-week “blanking period”. Following this study, a number of groups have reported their experience in utilizing SBRT to treat cardiac arrhythmias. However, despite its acute effectiveness, little is known about the long-term survival. Indeed, a collateral dose received by the surrounding anatomic structures may result in late effects. Further, existing approaches of cardiac radioablation typically involve planning target volumes that are enlarged to account for both cardiac and respiratory motion. This unnecessarily endangers healthy tissue. While cardiac motion can be accounted for by introducing a margin on the order of millimetres, respiratory motion often encompasses several centimetres.

One strategy for minimizing collateral dosing involves image guidance during treatment. The premise is that accounting for intrafraction motion should reduce the need for expanded target volumes, thereby limiting exposure to the surrounding healthy anatomy. For instance, known investigations have used orthogonal MRI planes for 3D target localization, and MRI-guided cardiac radioablation was used clinically to treat sustained VT. Additionally, robotically-guided radiosurgery was used for the creation of ablation lesions. However, the benefits of these approaches are yet to be demonstrated clinically. Moreover, both MRI-guidance and robotic-guidance require specialized systems that are not available in most clinical settings.

The use of multi-leaf collimator (MLC) tracking in radioablation treatments for AF has also been tested. However, this approach utilized implanted electromagnetic transponder beacons to provide a real-time motion signal. Implantation requiring surgery undermines the non-invasive nature of cardiac radioablation. Further, implantation is often a costly procedure, requiring the purchase of, and training for dedicated, specialised systems.

Therefore, there is a pressing need for effective and accessible therapies for the treatment of cardiac arrhythmias.

### SUMMARY

The present invention provides a method for x-ray guided cardiac radioablation, which can be implemented on a standard linear accelerator. The example method disclosed herein provides directly, precisely controlled x-ray guided cardiac radioablation that accurately targets the substrates of cardiac ablation while minimizing doses to healthy tissue. The example method uses one or more diaphragm tracking algorithms to account for respiratory motion during treatment.

At least a portion of the method may be performed pre-treatment while another portion of the method is performed during a treatment. For example, before a treatment, medical images of a patient’s diaphragm or respiratory surrogate, heart, and/or target are segmented by a computer system using a diaphragm tracking algorithm. The example computer system, using the diaphragm tracking algorithm, next performs a peak-exhale to peak-inhale registration. During this pre-treatment portion, the diaphragm tracking algorithm causes the computer system to generate a respiratory motion model. Subsequently during a treatment, the diaphragm tracking algorithm causes the computer system to track the patient’s diaphragm using X-ray imaging. Based on the tracking system, the computer system, using the diaphragm tracking algorithm, estimates a target position for radioablation.

According to one non-limiting aspect of the present disclosure, there is provided a method for cardiac substructure tracking. In one embodiment, the method includes segmenting a patient’s diaphragm or respiratory surrogate, heart, and target, performing a peak-exhale to peak-inhale registration, generating respiratory motion model, tracking the patient’s diaphragm using X-ray imaging, and estimating a target position.

According to another non-limiting aspect of the present disclosure, there is provided system for cardiac substructure tracking. In one embodiment, the system includes a memory configured to store instructions, and one or more processors in communication with the memory. The one or more processors are configured to execute the instructions to segment a diaphragm or respiratory surrogate, heart and target, perform a peak-exhale to peak-inhale registration, generate respiratory motion model, track diaphragm using X-ray imaging, and estimate a target position.

According to another non-limiting aspect of the present disclosure, any of the features, functionality and alternatives described in connection with any one or more of FIGS. 1 to 7 may be combined with any of the features, functionality and alternatives described in connection with any other of FIGS. 1 to 7.

According to the present disclosure, a preferred outcome is that the system and method for cardiac substructure tracking can greatly reduce target volumes and healthy tissue exposure.

It should be understood that the outcomes described herein are not limited, and may be any of or different from the outcomes described in the present disclosure.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

Features of systems and methods for cardiac structure tracking during ablative radiotherapy as described herein may be better understood by reference to the accompanying drawings in which:

FIG. 1 illustrates a pictographic representation of a clinical workflow for a method of tracking cardiac substructure, according to an embodiment of the present disclosure.

FIG. 2 illustrates graphs for tracking performance for a method using an algorithm along the LR (top), SI (middle) and AP (bottom) axes with the ground truth and predicted traces, according to an embodiment of the present disclosure.

FIG. 3 illustrates a sample tracking frame depicting the ground truth and predicted positions of the left atrium, according to an embodiment of the present disclosure.

FIG. 4 illustrates graphs for tracking performance for a first minute of a simulation with a lowest 3D error, including example projections at lateral and ventral views, according to an embodiment of the present disclosure.

FIG. 5 illustrates graphs for tracking performance for a first minute of a simulation with a greatest 3D error, including example projections at lateral and ventral views, according to an embodiment of the present disclosure.

FIG. 6 illustrates graphs for tracking performance for a first minute of a simulation with the with the lowest target coverage, including example projections at lateral and ventral views, according to an embodiment of the present disclosure.

FIG. 7 illustrates an example system for cardiac structure tracking, according to an example embodiment of the present disclosure.

### DETAILED DESCRIPTION

The terms “a,” “an,” “the” and similar referents used in the context of describing the invention (especially in the context of the following claims) are to be construed to cover both the singular and the plural, unless otherwise indicated herein or clearly contradicted by context.

All methods described herein can be performed in any suitable order unless otherwise indicated herein or otherwise clearly contradicted by context.

The use of any and all examples, or exemplary language provided herein is intended merely to better illuminate the invention and does not pose a limitation on the scope of the invention otherwise claimed. No language in the specification should be construed as indicating any non-claimed element essential to the practice of the invention.

The embodiments will now be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements.

The present invention focuses toward the development of technologies that accurately target the substrates of cardiac ablation while minimizing dose to healthy tissue. Additionally, the invention provides a markerless method for x-ray guided cardiac radioablation. In particular, a direct diaphragm tracking algorithm is leveraged to account for respiratory motion during treatment. Indeed, while the pulsatile motion of cardiac substructures typically spans several millimetres, respiratory motion is often on the order of several centimetres. Therefore, respiration monitoring will account for the bulk of intrafraction motion, and thus enable the use of significantly reduced treatment margins.

According to an embodiment disclosed herein, there is provided a clinical workflow defined by one or more algorithms for x-ray guided cardiac radioablation and a method which utilizes diaphragm tracking to account for respiratory motion during treatment. The method is validated by using the left atrium as a prospective target on a digital phantom, for which there is objective ground truth for quantitative analysis.

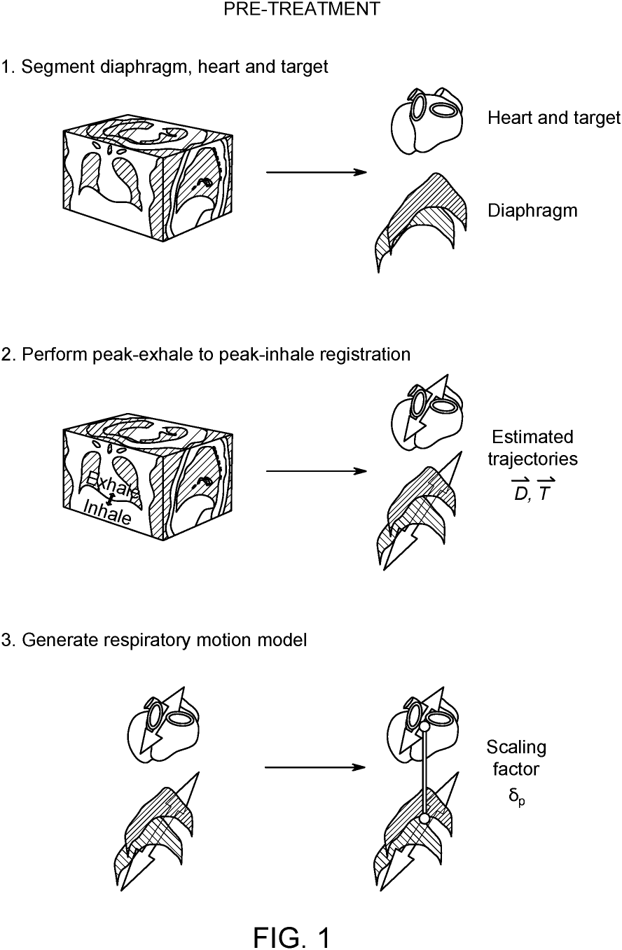

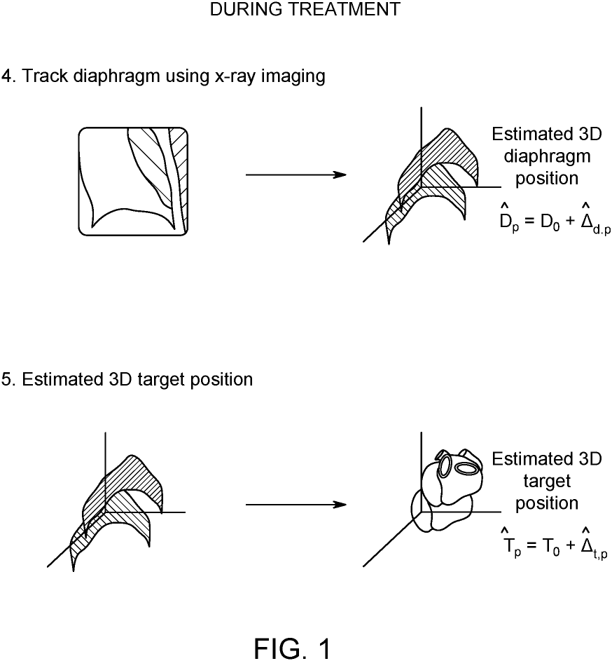

FIG. 1 is pictographic representation of the clinical method for x-ray guided cardiac radioablation. The method may be defined by one or more instructions stored in a memory device. The instructions, in aggregate, define a diaphragm tracking algorithm. Execution of these instructions by a computer system cause the computer system to perform the operations described herein.

The clinical method may include a pre-treatment step and a during-treatment step. The pre-treatment step may include but not limited to (1) segmenting a patient’s diaphragm, heart, and target; (2) performing peak-exhale to peak-inhale registration; and (3) generating a respiratory motion model. The during-treatment step may include but not limited to (4) tracking the patient’s diaphragm using x-ray imaging and (5) estimating a 3D target position for x-ray guided cardiac radioablation.

According to an embodiment of the present disclosure, a workflow of the tracking method implemented by the diaphragm tracking algorithm disclosed herein includes steps 1-3, which occur pre-treatment, and steps 4-5 which occur during-treatment:
1. Segment a diaphragm or other respiratory surrogate, heart and target;
2. Perform peak-exhale to peak-inhale registration;
3. Generate respiratory motion model;
4. Track diaphragm using x-ray imaging;
5. Estimate a target position (one or two or three dimensional target position).

### Segmentation

At step 1, a computer system using a diaphragm tracking algorithm is configured to automatically segment a patient’s diaphragm. In other embodiments, the computer system using the diaphragm tracking algorithm diaphragm enables the diaphragm to be segmented by a clinician or other qualified medical professional. The computer system is configured to segment the diaphragm by analysing medical images, such as computed tomography (CT) images. The diaphragm is segmented by identifying points of negative curvature at the lowermost boundaries of the left and right lungs separately. The heart is also automatically segmented or manually segmented by a clinician or other qualified medical professional by identifying the myocardium as well as the blood within each chamber. The target is automatically segmented or manually segmented by a clinician or other qualified medical professional using convex hulls to encompass contact points between the left atrium and pulmonary veins. Each structure was segmented using, for example, the peak-exhale four-dimensional computed tomography (4D-CT) images, as this typically exhibits the fewest respiratory motion artefacts.

### Peak-Exhale to Peak-Inhale Registration

At step 2, the computer system using the diaphragm tracking algorithm is configured to perform peak-exhale to peak-inhale registration. The trajectories of respiratory motion for the diaphragm or other respiratory surrogate and heart are estimated by rigidly registering each segment at peak-exhale to the peak-inhale 4D-CT images, assuming zero left-right (LR) motion and zero rotation. As the target represents a cardiac substructure, the trajectory of respiratory motion for the heart is used to determine that of the target. Therefore, the computer system generates registration between peak inhale to peak-exhale or between any two phases of a 4D-CT using motion vectors for the diaphragm and target of D̅ = (0 d_{SI} d_{AP}) and T̅ = (0 t_{SI} t_{AP}) respectively, where d_{SI}, t_{SI} represent the magnitudes of motion along the superior-inferior (SI) axis and d_{AP}, t_{AP} represent the magnitudes of motion along the anterior-posterior (AP) axis.

### Modelling Respiratory Motion

At step 3, the computer system using the diaphragm tracking algorithm is configured to model the previously registered respiratory motion. The computer system may use any position along the estimated trajectories for modelling by scaling the relative magnitudes of motion along at least one of the SI and AP axes. For instance, the extent of respiratory motion for the diaphragm at projection p can be estimated by:

$${\hat{Δ}}_{d , p} = \left(0 , {δ}_{p} ⋅ {d}_{S I} , {δ}_{p} ⋅ {d}_{A P}\right)$$

where δₚ is a scaling factor, for which values of 0 and 1 correspond to the peak-exhale and peak-inhale positions respectively. Additionally, this formulation enables the estimation of 3D diaphragm position, for any projection p, by:

$${\hat{D}}_{p} = {D}_{0} + {\hat{Δ}}_{d , p}$$

where D₀ is the 3D position of the diaphragm at peak-exhale. As such, the optimal value of δₚ can be determined via diaphragm tracking.

### Diaphragm Tracking

At step 4, the computer system using the diaphragm tracking algorithm is configured to perform diaphragm tracking. Briefly, each 3D diaphragm segment is forward-projected to generate 2D diaphragm maps. This is performed at increments of 0.5° as this was found to yield sufficient angular resolution. During treatment, the optimal value of δₚ is determined by shifting angle-matched 2D diaphragm maps along the estimated trajectory of diaphragmatic motion or motion of any other respiratory surrogate. This is achieved for each projection individually by using a modified maximum gradient algorithm.

### Respiratory Motion Estimation

At step 5, the computer system using the diaphragm tracking algorithm is configured to estimate a target position by estimating respiratory motion. By presuming that the respiratory component of target motion is directly proportional to that of diaphragmatic motion, the extent of target motion at projection p can be estimated by:

$${\hat{Δ}}_{t , p} = \left(0 , {δ}_{p} ⋅ {t}_{S I} , {δ}_{p} ⋅ {t}_{A P}\right)$$

Similarly, 3D target position at projection p can be estimated by:

$${\hat{T}}_{p} = {T}_{0} + {\hat{Δ}}_{t , p}$$

where T₀ is the 3D position of the target at peak-exhale.

### Digital Phantoms

In an embodiment, a 4D extended cardiac-torso (XCAT) phantom was used to generate imaging data with realistic internal motion as well as highly detailed and varied anatomies (Table 1). In particular, individual traces from the Combined measurement of ECG, Breathing and Seismocardiogram (CEBS) database were used to dictate cardio-respiratory motion for each phantom. These data were pre-processed to generate 17 traces 10 minutes in length. To evaluate the effect of diaphragmatic displacement on tracking accuracy, each phantom was randomly allocated to a cohort with maximum diaphragm motion amplitude set to 5, 10, or 20 mm. These cohorts reflect the approximate range of diaphragmatic displacement one standard deviation above and below average. Additionally, correspondence between the SI position of the diaphragm and the AP position of chest wall was set to 1:1 in all cases to reflect previous observations. Random allocations were achieved using the random permutation function in MATLAB. (The MathWorks, Inc., Natick, MA).

TABLE 1

Phantom characteristics. kg means kilograms; cm means centimetres; mm means millimetres

| Phantom | Age | Gender | Weight (kg) | Height (cm) | Diaphragm displacement (mm) |
|---|---|---|---|---|---|
| 1 | 56 | F | 70 | 167 | 5 |
| 2 | 54 | F | 87 | 161 | 5 |
| 3 | 52 | M | 61 | 173 | 5 |
| 4 | 62 | F | 79 | 160 | 10 |
| 5 | 41 | M | 100 | 177 | 10 |
| 6 | 67 | M | 103 | 182 | 10 |
| 7 | 63 | F | 81 | 153 | 20 |
| 8 | 50 | M | 120 | 178 | 20 |
| 9 | 34 | M | 104 | 188 | 20 |

### Pre-Treatment Imaging

4D-CT imaging was simulated using a 1-minute section from each 10-minute trace. Each respiratory trace was segmented into 10 discrete respiratory bins and detailed anatomic volumes were generated at a rate of 10.5 Hz. Volumes generated at peak-exhale and peak-inhale were averaged to produce the peak-exhale and peak-inhale 4D-CT respectively.

### Intrafraction Imaging

Intrafraction imaging was simulated using a 5-minute section from each 10-minute trace, which did not overlap with that used during four-dimensional computed tomography (4D-CT) imaging. Imaging was simulated over two treatment arcs by generating anatomic volumes at a rate of 10.5 Hz. Projections were acquired for each volume via Radon transform. This resulted in 3150 projections and volumes.

### Planning and Ground-Truth Target Volumes

Planning target volumes were generated for each patient by segmenting the left atrium on the peak-exhale 4D-CT. This was achieved by identifying points corresponding to the left atrial myocardium as well as the blood within this chamber and, subsequently, defining a convex hull that encompassed these points. Similarly, ground-truth target volumes were generated for each intrafraction volume by identifying points corresponding to the left atrial myocardium as well as blood within the chamber.

### Evaluating Tracking Performance

During intrafraction imaging, Δ̂_{t,p} was used to rigidly translate the planning target volume. Centres-of-mass for the shifted planning target volume and the ground-truth target volume were recorded as the estimated ground-truth 3D target positions respectively.

Tracking performance was evaluated using three metrics. Firstly, geometric error was recorded for each projection by computing the difference between the estimated 3D target positions and the ground-truth 3D target positions. Secondly, similarities between the planning and ground-truth target volumes was recorded using Dice similarity coefficients. Lastly, volumetric coverage of the ground-truth target volumes were recorded for planning target volumes with isotropic expansion of 1, 2 and 3 mm.

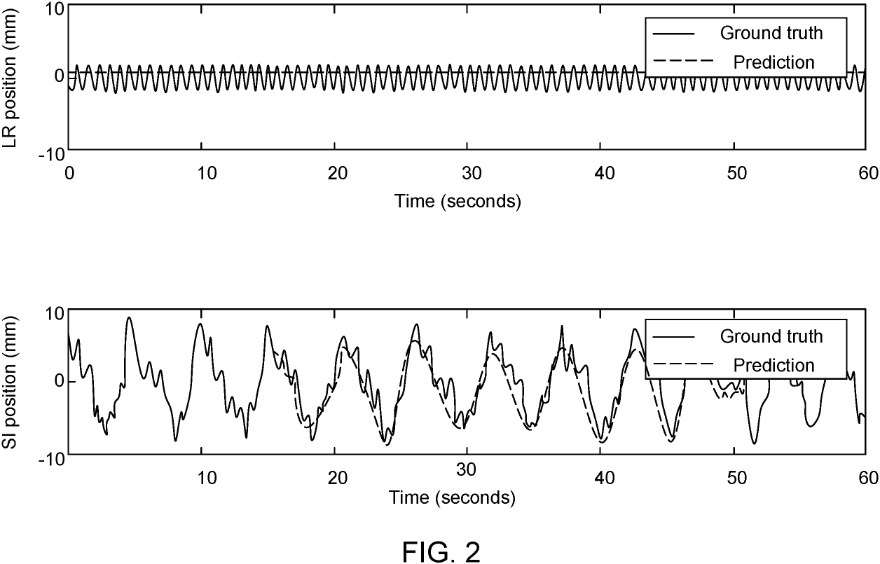

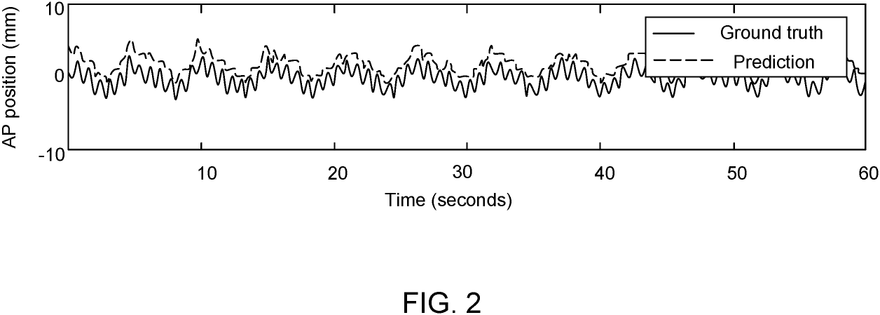

FIG. 2 illustrates the tracking performance for the algorithm along the left-right (LR), superior-inferior (SI) and anterior-posterior (AP) axes with the ground truth and predicted traces for the first minute. As a result, Mean geometric error along the left-right (LR), superior-inferior (SI) and anterior-posterior (AP) axes was -0.64, 0.56 and -1.90 mm respectively. Mean dice similarity between predicted and ground truth volumes was 0.84. Volumetric coverage of the ground truth volumes was > 89%, > 96% and > 99% for planning target volumes with isotropic expansions of 1, 2 and 3 mm respectively.

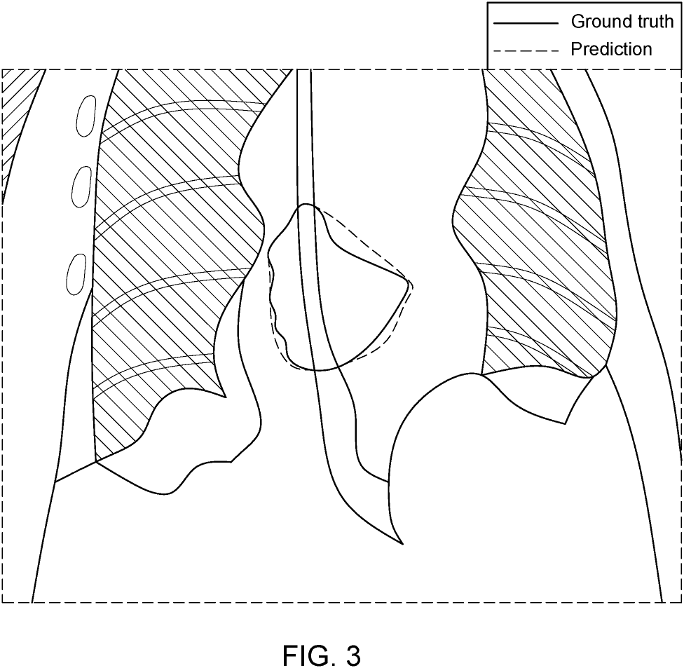

FIG. 3 illustrates a sample tracking frame depicting the ground truth and predicted positions of the left atrium. The prediction result (dashed edge) by using the algorithm has a good match with the ground truth (solid edge). Comparing centroid positions along the superior-inferior axis, there is good agreement between the target and ground-truth volumes. These results suggest that diaphragm tracking is able to be used to account for respiratory motion during cardiac radioablation. The tracking method provides a significant advancement toward the safe and effective use of radiotherapy for the treatment of refractory cardiac arrhythmias.

### Comparing Simulations With and Without Real-Time Image Guidance

In some embodiments, for simulations with real-time image guidance, pre-treatment segmentation of the target, heart, and diaphragm was performed on the peak-exhale 4D-CT, as this phase typically exhibits the fewest motion artefacts. One major advantage of a 4D-XCAT phantom is that every voxel is labelled (via exact intensity values) according to the corresponding anatomical structure. Therefore, a cardiac internal target volume (ITVc) is defined for each phantom by identifying voxels corresponding to the relevant substructure over every cardiac phase and, subsequently, defining a convex hull encompassing these points. The diaphragm was segmented by identifying points of negative curvature at the lowermost boundaries of the left and right lungs separately. The heart was segmented by identifying voxels corresponding to the myocardium as well as the blood within each chamber.

For simulations without real-time image guidance, target segmentation emulated that used in the prospective phase ½ ENCORE-VT trial (Knutson et al. 2019). That is, a combined respiratory and cardiac internal target volume (ITV_{R+C}) was segmented by identifying voxels corresponding to the left atrium over every cardiac and respiratory phase and defining a convex hull encompassing these points. ITVc and ITV_{R+C} were both expanded using a 3 mm isotropic margin to yield the planning target volumes (PTV_{C}) and (PTV_{R+C}) respectively. This margin expansion is selected based on a planning study, which proposed 3 mm as the maximum tolerable margin to ensure adequate sparring of critical structures. It should be noted that, while PTV_{R+C} remains static as in conventional radiotherapy, PTVc is dynamically shifted in this embodiment for simulations with real-time image guidance. Ground-truth target volumes were generated by identifying voxels corresponding to the left atrium on each intrafraction volume.

During simulated intrafraction imagingΔ̅_{t,p} was used to dynamically shift PTVc while PTVR+C remained unshifted. Target, heart, and diaphragm contours were then projected onto each tracking frame and used to create videos for real-time visualization. Treatment simulations with and without real-time image guidance were compared using four metrics. First, the difference in sizes of PTVc and PTV_{R+C} are recorded. Second, mean volumetric coverage of the ground-truth target volumes was recorded for PTVc and PTV_{R+C}. Third, minimum volumetric coverage of the ground-truth target volumes was recorded for PTVc and PTV_{R+C}. This metric is reported as the lowest coverage for any given tracking frame. Lastly, geometric error for scenarios with and without real-time image guidance was estimated by recording differences in the 3D positions of the centres-of-mass for the shifted PTVc and unshifted PTV_{R+C} with the ground-truth target volume.

Paired-sample Student’s t-tests were performed (at a significance level of 0.05) for simulations with and without real-time image guidance to determine whether differences in target volume size, mean volumetric coverage, minimum volumetric coverage and geometric error were statistically significant.

Comparing simulations with and without real-time image guidance, the sizes of PTVc and PTV_{R+C} ranged between 141 – 196 cc and 165 – 239 cc respectively with differences ranging between 11 – 24%. These differences were found to be statistically significant (p < 0.001). These results are summarised in Table 2 below.

TABLE 2

Differences in planning target volume size for simulations with (PTVc) and without (PTV_{R+C}) real-time image guidance

| Phantom | PTVc(cc) | PTV_{R+C} (cc) | Difference (cc) | Difference (%) |
|---|---|---|---|---|
| 1 | 170 | 201 | 31 | 15 |
| 2 | 141 | 165 | 24 | 15 |
| 3 | 179 | 202 | 23 | 11 |
| 4 | 174 | 207 | 33 | 16 |
| 5 | 166 | 217 | 51 | 24 |
| 6 | 196 | 239 | 43 | 18 |
| 7 | 178 | 232 | 54 | 23 |
| 8 | 179 | 224 | 45 | 20 |

Mean volumetric coverage ranged between 98.6 - 100.0% and 99.6 - 100.0% for the shifted PTVc and unshifted PTV_{R+C} respectively. Differences in mean volumetric coverage were not statistically significant (p = 0.35). Similarly, minimum volumetric coverage ranged between 94.1 - 100.0% and 96.1 - 100.0% for the shifted PTVc and unshifted PTV_{R+C} respectively. These differences were also statistically insignificant (p = 0.35). Phantom 1 was the only case in which the planning target volumes did not completely envelop the ground-truth target volumes across all simulations. These results are summarized below in Tables 3 and 4.

TABLE 3

Mean volumetric coverage (%) for simulations with (shifted PTVc) and without (unshifted PTV_{R+C}) real-time image guidance

| Phantom | Shifted PTVc (cc) | Unshifted PTV_{R+C} (cc) |
|---|---|---|
| 1 | 98.6 | 99.6 |
| 2 | 100.0 | 100.0 |
| 3 | 100.0 | 100.0 |
| 4 | 100.0 | 100.0 |
| 5 | 100.0 | 100.0 |
| 6 | 100.0 | 100.0 |
| 7 | 100.0 | 100.0 |
| 8 | 100.0 | 100.0 |

TABLE 4

Minimum volumetric coverage (%) for simulations with (shifted PTVc) and without (unshifted PTV_{R+C}) real-time image guidance

| Phantom | Shifted PTVc (cc) | Unshifted PTV_{R+C} (cc) |
|---|---|---|
| 1 | 94.1 | 96.1 |
| 2 | 100.0 | 100.0 |
| 3 | 100.0 | 100.0 |
| 4 | 100.0 | 100.0 |
| 5 | 100.0 | 100.0 |
| 6 | 100.0 | 100.0 |
| 7 | 100.0 | 100.0 |
| 8 | 100.0 | 100.0 |

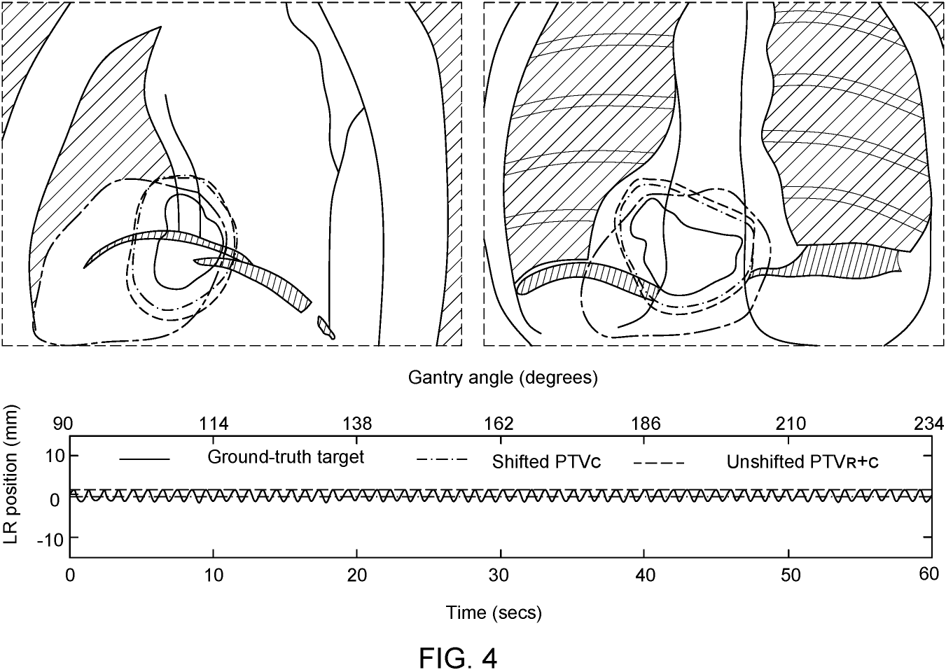

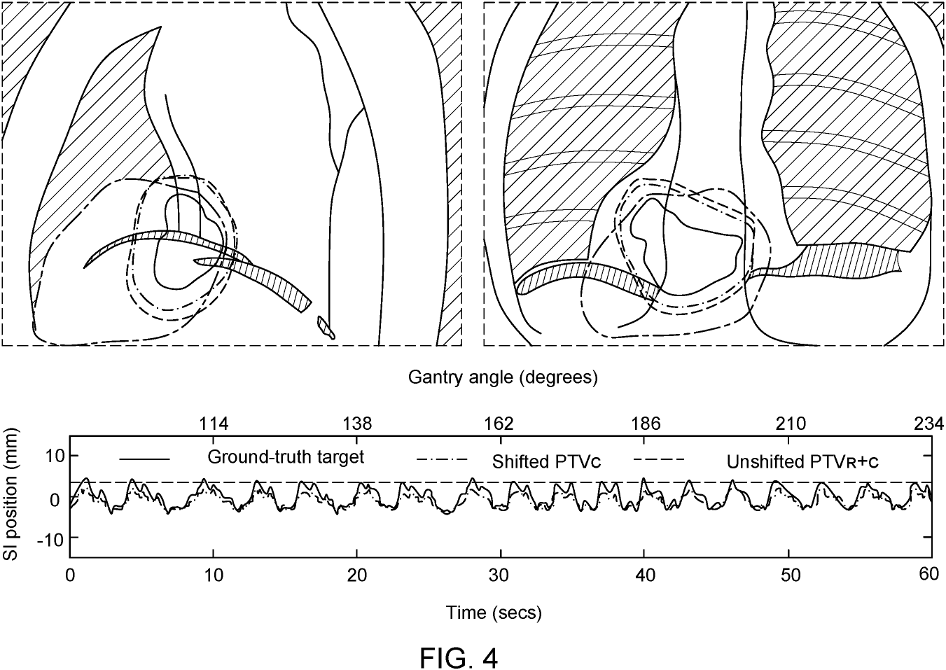

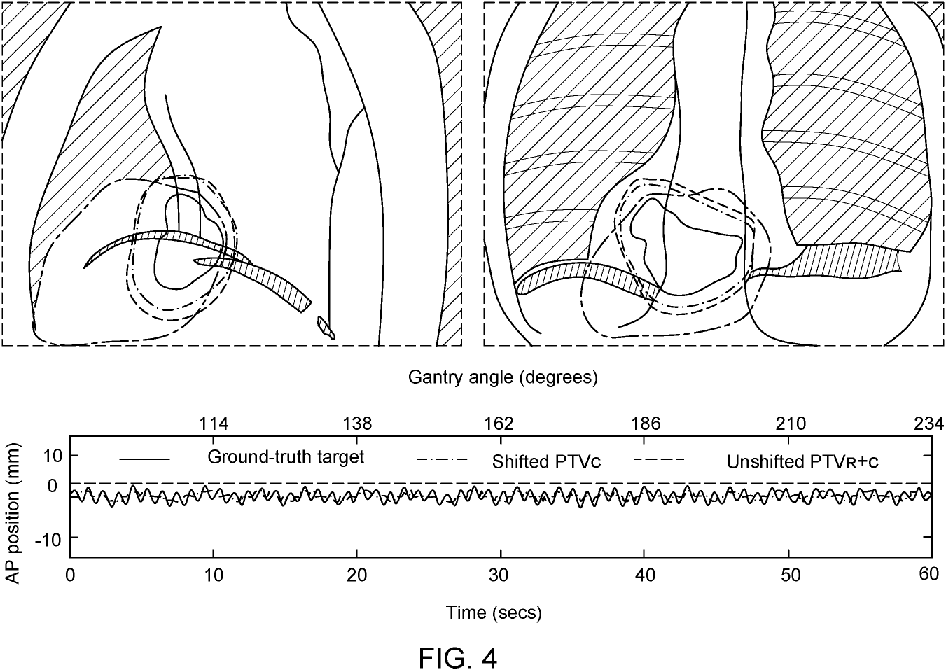

For simulations with and without real-time image guidance, mean 3D geometric error ranged between 1.8 - 6.0 mm and 3.2 - 9.7 mm respectively. Differences in mean 3D geometric error were statistically significant (p = 0.039). These results are summarized below in Tables 5 and 6. The lowest 3D error for simulations with real-time image guidance was observed for Phantom 2 with mean errors of 0.1 ± 0.9, 0.9 ± 0.8 and -0.1 ± 1.3 mm along the LR, SI and AP axes respectively. Tracking performance for the first minute of this simulation is depicted in FIG. 4.

FIG. 4 illustrates graphs and images for tracking performance for a first minute of a simulation with the lowest 3D error (Phantom 2), including example projections at lateral and ventral views overlaid with ground-truth target, shifted PTVc, and unshifted PTV_{R+C} shown in solid lines. Further, heart and diaphragm positions are shown in solid lines. Motion traces for the ground-truth target, shifted PTVc and unshifted PTV_{R+C} centroid positions are also shown.

TABLE 5

Mean and standard deviation of the geometric errors across simulations with real-time image guidance. Mean ± SD (mm)

| Phantom | LR | SI | AP | 3D |
|---|---|---|---|---|
| 1 | 0.1±1.1 | 2.6±0.6 | -0.1±1.3 | 3.1±0.6 |
| 2 | 0.1±1.9 | 0.9±0.8 | -0.1±1.3 | 1.8±0.7 |
| 3 | 0.5±1.0 | 4.1±0.8 | 0.6±1.3 | 4.4±1.0 |
| 4 | -1.3±1.0 | 1.6±0.5 | -2.1±1.5 | 3.3±1.2 |
| 5 | -0.6±1.3 | 0.1±0.9 | -1.9±0.9 | 2.5±1.0 |
| 6 | -2.1±1.0 | -2.9±0.7 | -4.2±1.4 | 5.7±1.6 |
| 7 | -2.0±1.0 | -2.0±0.7 | -5.2±1.4 | 6.0±1.7 |
| 8 | 0.0±1.1 | 3.8±1.4 | -0.0±1.3 | 4.2±1.3 |
| Overall | -0.7±1.4 | 1.0±2.5 | -1.6±2.4 | 3.9±1.8 |

TABLE 6

Mean and standard deviation of the geometric errors across simulations without real-time image guidance. Mean ± SD (mm)

| Phantom | LR | SI | AP | 3D |
|---|---|---|---|---|
| 1 | -0.7±1.1 | -1.5±2.3 | -1.5±1.3 | 3.2±1.7 |
| 2 | -1.5±0.9 | -3.5±2.2 | -3.3±1.3 | 5.2±2.2 |
| 3 | -0.4±1.0 | -0.8±2.6 | -1.2±1.4 | 3.3±1.1 |
| 4 | -2.6±1.0 | -2.7±2.4 | -3.8±1.5 | 5.7±2.2 |
| 5 | 0.9±1.3 | -7.7±4.0 | -2.3±1.3 | 8.3±3.9 |
| 6 | 0.0±1.0 | -3.7±4.8 | -0.7±1.8 | 5.2±3.7 |
| 7 | 0.5±1.0 | 9.0±3.9 | 1.8±1.6 | 9.4±3.8 |
| 8 | -2.2±1.1 | -7.5±4.5 | -5.1±1.6 | 9.7±4.3 |
| Overall | -1.2±2.2 | -2.1±6.1 | -2.0±2.5 | 6.3±2.5 |

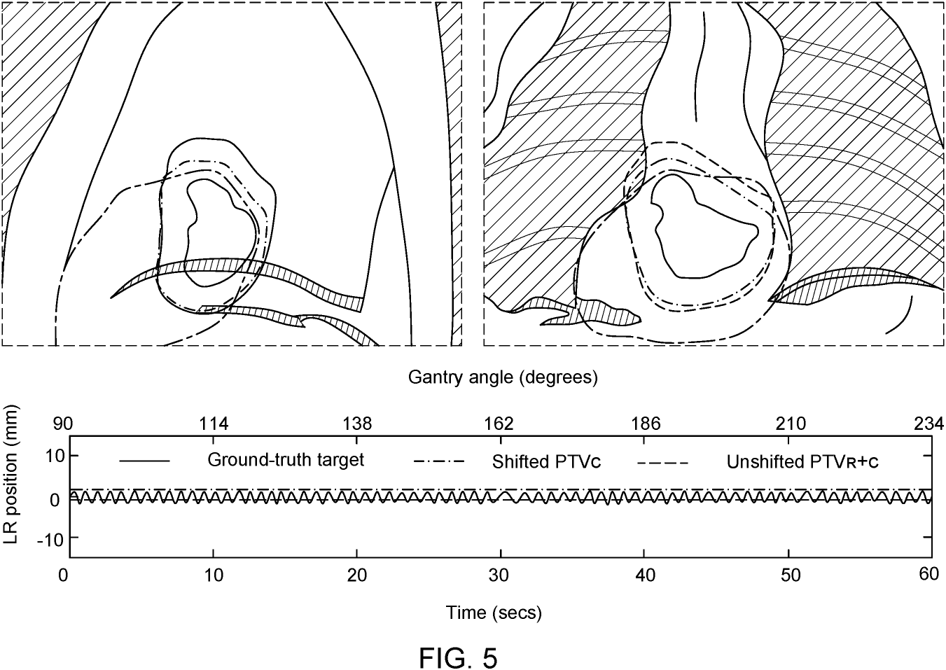

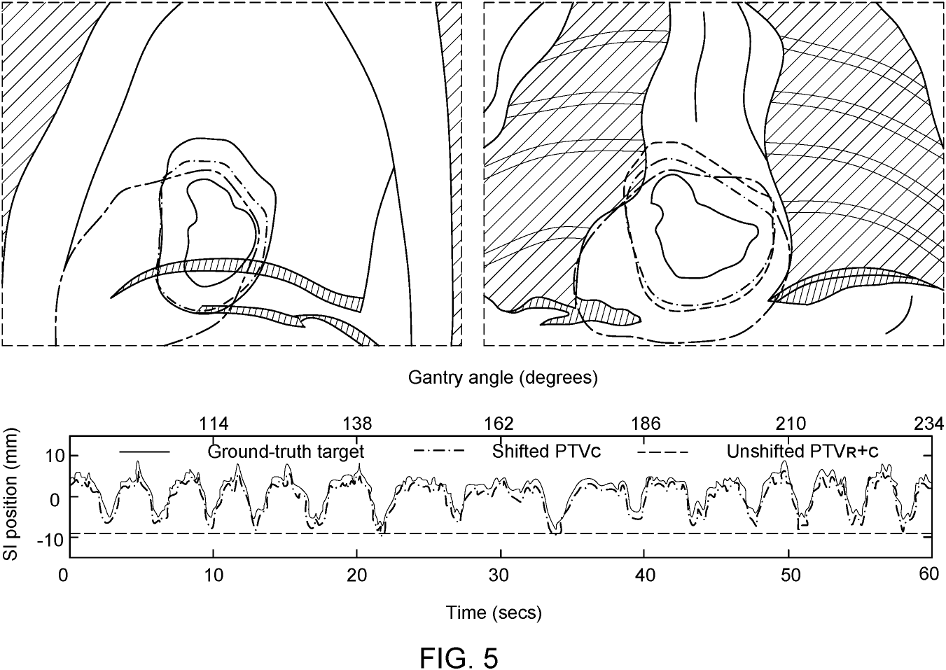

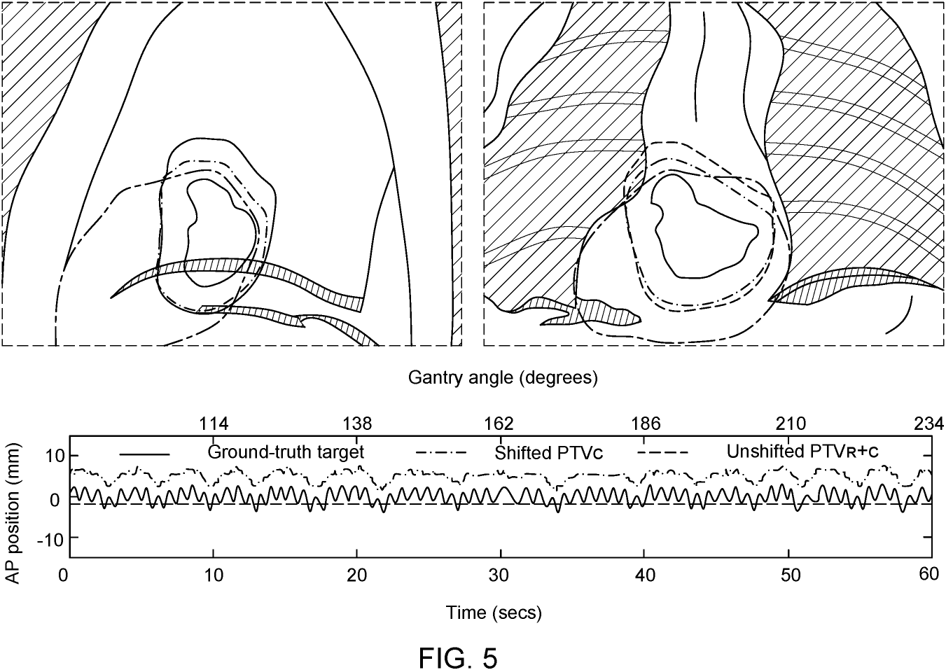

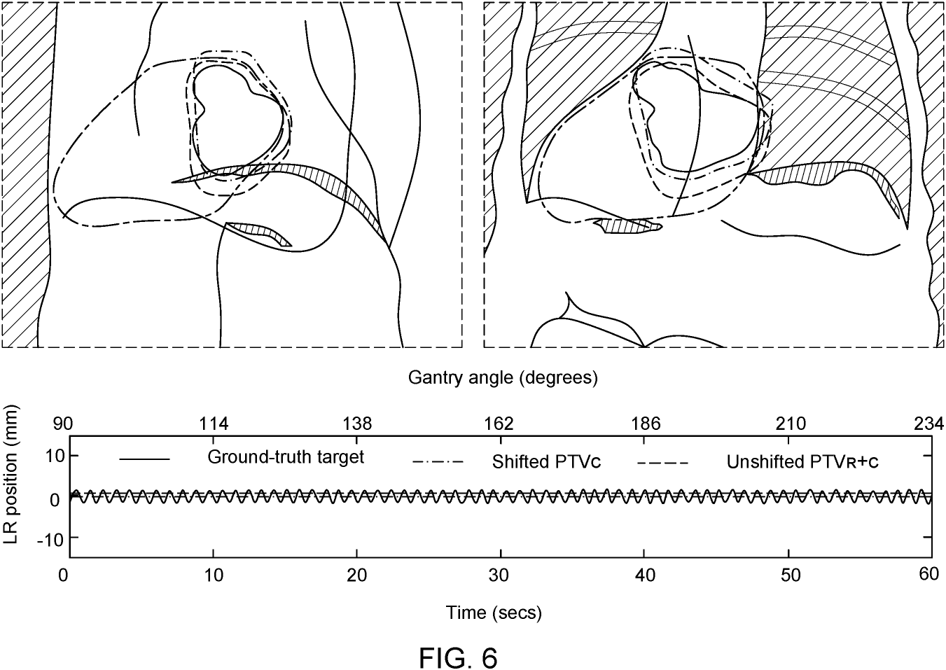

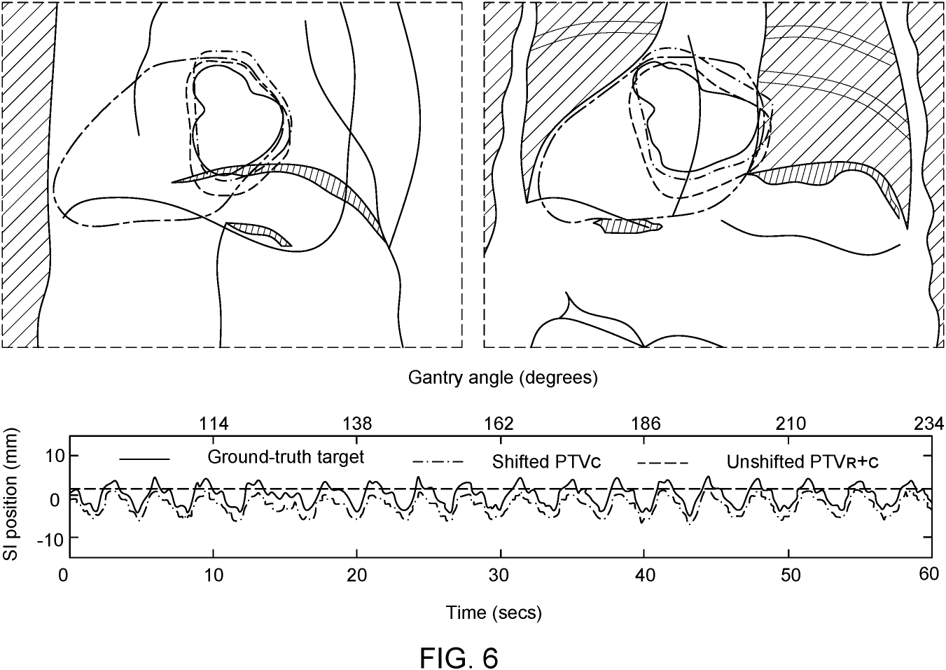

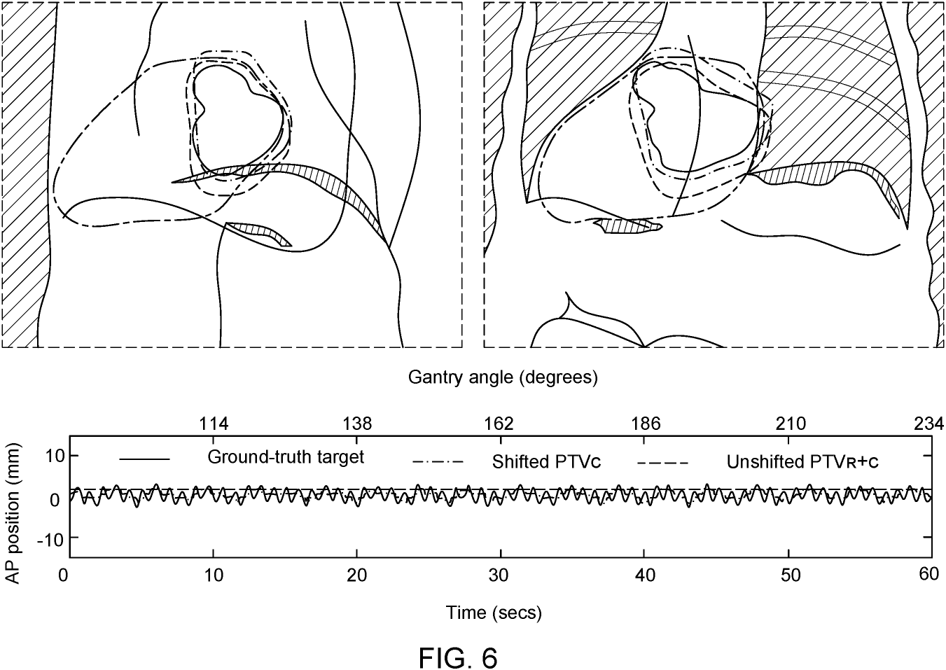

The highest 3D error for simulations with real-time image guidance was observed for Phantom 7 with mean errors of -2.0 ± 1.0, -2.0 ± 0.7 and -5.2 ± 1.4 mm along the LR, SI and AP axes respectively. Tracking performance for the first minute of this simulation is depicted in FIG. 5, which indicates that geometric errors arose due to consistent offsets along LR, SI and AP axes. Similarly, as shown in FIG. 6, an offset in the SI axis was observed for Phantom 1, which was the only simulation with less than 100% target coverage. FIG. 5 shows graphs and images for tracking performance for the first minute of the highest 3D error (Phantom 7), including example projections at lateral and ventral views overlaid with ground-truth target, shifted PTVc, and unshifted PTV_{R+C} shown in solid lines. Further, heart and diaphragm positions are shown in solid lines. Motion traces for the ground-truth target, shifted PTVc and unshifted PTV_{R+C} centroid positions are also shown. FIG. 6 shows graphs and images for tracking performance for the first minute of the simulation with the lowest target coverage (Phantom 1), including example projections at lateral and ventral views overlaid with ground-truth target, shifted PTVc, and unshifted PTV_{R+C} shown in solid lines. Further, heart and diaphragm positions are shown in solid lines. Motion traces for the ground-truth target, shifted PTVc and unshifted PTV_{R+C} centroid positions are also shown.

### Example System

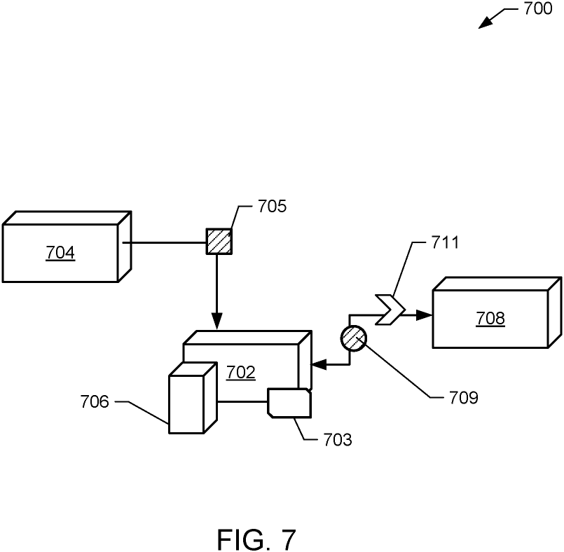

FIG. 7 illustrates an example system 700 for cardiac structure tracking, according to an example embodiment of the present disclosure. The example system 700 includes a computer system 702 including machine-readable instructions 703. Execution of the machine-readable instructions 703 cause the computer system 702 to perform the operations described herein. For example, the machine-readable instructions 703 define one or more diaphragm tracking algorithms.

The computer system 702 is communicatively coupled to a first medical device 704 via a directed connector or via a network. The first medical imaging device 704 may include a CT imaging device for recording 4D-CT data 705. The first medical imaging device 704 may include any imaging device configured for recording time-lapsed volumetric data of a patient’s diaphragm.

The computer system 702 is configured to segment (or provide for the segmentation) the patient’s diaphragm, heart, and/or target. The computer system 702 is also configured to determine trajectories of the patient’s diaphragm, heart, and/or target from end-inhale to end-exhale. The computer system 702 may perform peak-exhale to peak-inhale registration for determining the trajectories.

The computer system 702 is configured to generate a respiratory motion model 706 using the determined trajectories of the patient’s diaphragm, heart, and/or target and/or the peak-exhale to peak-inhale registration. The respiratory motion model 706 defines a relative contribution of the patient’s diaphragm to target motion. The respiratory motion model 706 may be determined by computing the magnitudes of motion over each trajectory.

After the respiratory motion model 706 is created, the patient may undergo an x-ray guided cardiac radioablation treatment. The computer system 702 is communicatively coupled to a second medical imaging device 708, which may include an x-ray imaging device and/or a multi-leaf collimator (MLC). The computer system 702 receives, for example x-ray images 709 from the second medical imaging device 708. For each kV projection, the computer system 702 is configured to estimate a 3D position of the diaphragm using diaphragm tracking provided by the respiratory motion model 706. The computer system 702 is configured to use the diaphragm tracking and/or the respiratory motion model 706 to determine a 3D position of a target 711 for cardiac radioablation treatment. The computer system 702, in some embodiments, transmits the 3D position of the target 711 to the second medical imaging device 708, thereby causing the second medical imaging device to provide cardiac radioablation treatment to a smaller area of patient tissue corresponding to the substrates of cardiac ablation. This targeted treatment minimizes the dose to healthy tissue of the patient.

### Conclusion

An advantage of the clinic method of the present disclosure is that it can greatly reduce target volumes and healthy tissue exposure. The Extended Cardiac-Torso (XCAT) digital phantom is used to create detailed anatomical volumes. Cardiac and respiratory motion are driven using traces acquired for a healthy volunteer with diaphragm motion set to 0.5, 1 or 2 cm. The clinical workflow includes stages post 4D-CT acquisition (1-2) and during kV imaging (3-4):
1. The diaphragm and target are segmented and their trajectories from end-inhale to end-exhale are estimated using a 4D-CT;
2. The relative contribution of diaphragm to target motion is computed by comparing the magnitudes of motion over each trajectory;
3. For each kV projection, the 3D position of the diaphragm is estimated using diaphragm tracking;
4. The 3D position of the diaphragm is used to estimate the 3D position of target.

In scenarios where cardiac substructure tracking is used, the target is defined using a convex hull which encompassed the position of the pulmonary vein antrum (PVA) on the end-exhale phase 4D-CT. Where substructure tracking is not used, the target corresponds to the position of the PVA over all respiratory phases. In both scenarios, a 3 mm isotropic margin is used to account for pulsatile cardiac motion.

The terminology used herein is for the purpose of describing particular example embodiments only and is not intended to be limiting. As used herein, the terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for cardiac substructure tracking, comprising:
segmenting, via a computer system, a diaphragm or respiratory surrogate, heart, and target;
performing, via the computer system, a peak-exhale to peak-inhale registration;
generating, via the computer system, a respiratory motion model;
tracking, via the computer system, the diaphragm using X-ray imaging; and
estimating, via the computer system, a target position for an x-ray guided cardiac radioablation treatment.

2. The method of claim 1, wherein the diaphragm is segmented by the computer system enabling identification of points of negative curvature at lowermost boundaries of left and right lungs.

3. The method of claims 1 or 2, wherein trajectories of the diaphragm or respiratory surrogate and target are configured to be estimated using a four-dimensional computed tomography (4D-CT).

4. The method of claims 1, 2, or 3, wherein the heart is segmented by identifying myocardium and blood within each chamber.

5. The method of claims 1, 2, 3, or 4, wherein the target is segmented by using convex hulls to encompass contact points between a left atrium and pulmonary veins.

6. The method of claims 1 or 5, wherein the registration between peak-exhale to peak-inhale or between two phases of a 4D-CT is configured to yield motion vectors for the diaphragm and target of D [symbol] = (0 d_{SI} d_{AP}) and [symbol] T = (0 T_{SI} t_{AP}) respectively, where d_{SI}, T_{SI} represent magnitudes of motion along a superior-inferior axis and d_{AP}, t_{AP} represent magnitudes of motion along an anterior-posterior axis.

7. The method of claim 6, wherein a respiratory motion is modelled by scaling relative magnitudes of motion along at least one of the superior-inferior axis and the anterior-posterior axis.

8. The method of claim 6, wherein a respiratory motion is modelled for the diaphragm at a projection p by estimating:
$${\hat{Δ}}_{d , p} = \left(0 , {δ}_{p} ⋅ {d}_{S I} , {δ}_{p} ⋅ {d}_{A P}\right)$$
where δₚ is a scaling factor, for which values of 0 and 1 correspond to the peak-exhale and peak-inhale positions respectively.

9. The method of claim 8, wherein during the x-ray guided cardiac radioablation treatment, an optimal value of δₚ is determined by shifting angle-matched two-dimensional diaphragm maps along an estimated trajectory of diaphragmatic motion.

10. The method of claims 1 or 9, wherein a respiratory component of target motion is proportional to motion of the diaphragm or respiratory surrogate.

11. The method of claims 1, 9 or 10, wherein the target position includes at least one of a one dimensional target position, a two dimensional target position, or a three dimensional target position.

12. A system for cardiac substructure tracking, comprising:
a memory configured to store instructions; and
one or more processors in communication with the memory, the one or more processors configured to execute the instructions, causing the one or more processors to:
segment a diaphragm or respiratory surrogate, heart, and target,
perform a peak-exhale to peak-inhale registration,
generate respiratory motion model,
track diaphragm using X-ray imaging, and
estimate a target position for an x-ray guided cardiac radioablation treatment.

13. The system of claim 12, wherein the computer system is configured to segment the diaphragm for identifying points of negative curvature at lowermost boundaries of left and right lungs.

14. The system of claims 12 or 13, wherein trajectories of the diaphragm or respiratory surrogate and target are configured to be estimated using a four-dimensional computed tomography (4D-CT).

15. The system of claims 12, 13, or 14, wherein the heart is segmented by identifying myocardium and blood within each chamber, and
wherein the target is segmented by using convex hulls to encompass contact points between a left atrium and pulmonary veins.

16. The system of claims 12 or 15, wherein the registration between peak-exhale to peak-inhale or between two phases of a 4D-CT is configured to yield motion vectors for the diaphragm and target of [symbol] = (0 d_{SI} d_{AP}) and [symbol] = (0 t_{SI} t_{AP}) respectively, where d_{SI}, t_{SI} represent magnitudes of motion along a superior-inferior axis and d_{AP}, t_{AP} represent magnitudes of motion along an anterior-posterior axis.

17. The system of claim 16, wherein a respiratory motion is modelled by scaling relative magnitudes of motion along at least one of the superior-inferior axis and the anterior-posterior axis.

18. The system of claim 16, wherein a respiratory motion is modelled for the diaphragm at a projection p by estimating:
$${\hat{Δ}}_{d , p} = \left(0 , {δ}_{p} ⋅ {d}_{S I} , {δ}_{p} ⋅ {d}_{A P}\right)$$
where δₚ is a scaling factor, for which values of 0 and 1 correspond to the peak-exhale and peak-inhale positions respectively.

19. The system of claim 18, wherein during the x-ray guided cardiac radioablation treatment, an optimal value of δₚ is determined by shifting angle-matched two-dimensional diaphragm maps along an estimated trajectory of diaphragmatic motion.

20. The system of claims 12 or 19, wherein a respiratory component of target motion is proportional to motion of the diaphragm or respiratory surrogate.
